(19) United States
(12) Patent Application Publication
(10) Pub. No.: US 2011/0160851 A1
(43) Pub. Date: Jun. 30, 2011

(54) Keratoprosthesis

(75) Inventor: Wolfgang Mueller-Lierheim, Munich (DE)

(73) Assignee: Coronis GmbH, Muenchen (DE)

(21) Appl. No.: 12/746,403

(22) PCT Filed: Dec. 5, 2008
(86) PCT No.: PCT/EP08/10337

§ 371 Date: Aug. 3, 2010

(30) Foreign Application Priority Data
Dec. 6, 2007 (EP) 07023687.2

(51) Int. Cl. A61F 2/14

(52) U.S. Cl. 623/5.13

(57) ABSTRACT

A keratoprosthesis having a central optical part tightly connected to a peripheral haptic part both made of hydrophobic polymers. Smooth surfaces of the optical part are present where the surface oriented toward the eyelid has been rendered hydrophilic, whereas, the chemical nature of the surface oriented to the anterior chamber of the eye is such that it does not support fibrin adherence and membrane formation. The chemical nature of the textured haptic part is configured to promote the adherence of cells and eventually the vascular ingrowth.

### BACKGROUND AND SUMMARY OF THE INVENTION

The invention concerns a keratoprosthesis having a central optical part connected to a peripheral haptic part both made of hydrophobic material.

Opacification of the cornea of the human eye results in the loss of vision and finally blindness unless corrected by a corneal transplant. Conditions that may require corneal transplants include Keratoconus (a local steepening of the curvature of the cornea) if it cannot be corrected by special contact lenses, hereditary corneal failure (e.g. Fuchs endothelial dystrophy), extensive scarring and/or vascularization after infections (e.g. Herpes and trachoma) or penetrating injury, and corneal opacification after eye surgery including refractive surgery (LASIK). The most frequent surgical technology to restore vision is the replacement of the cornea by a human donor cornea in a penetrating keratoplasty. In this procedure which was first performed by Zirm in 1906 a circular part of the damaged cornea is removed and replaced by the respective part of a cadaver cornea which is sutured into the host cornea. In developed countries corneal transplantion using donor cornea provided by a network of corneal banks are the most common and successful operations in transplant surgery. More than 40.000 keratoplasties per year are performed in Europe and the United States each, with a continuous increase in recent years.

Recipients of corneal transplants generally require long-term local therapy with antibiotics, antiinflammatory and anti-rejection drugs. Depending on a number of circumstances the success rate (i.e. clear transplant six months after surgery) varies from more than 90 to less than 50 percent. Low success rates are associated with dry eyes, Herpes keratitis, corneal vascularization, recurring uveitis, acid burns, and traumatic anatomic structures of the anterior eye. The prognosis of keratoplasty also depends on the quality of the donor cornea including its storage and transport. There is a lack of donor corneas resulting in long waiting lists of patients in developed countries. Due to the lack of infrastructure there are no corneal banks in developing countries resulting in millions of treatable blind people in these countries.

The idea to replace the human cornea by alloplastic material dates back more than 200 years. In 1789 the French ophthalmologist Pellier de Quengsy proposed the implantation of a glass plate surrounded by a silver ring into the human cornea. In 1853 Nussbaum performed during his doctoral thesis in Munich experiments on rabbit eyes with a reported implant survival time of seven months. Six years later the Swiss surgeon Heusser implanted a glass plate into the cornea of a 19 year old who lost the implant after only three months. Between 1877 and 1887 von Hippel implanted seven corneal implants in human eyes with a maximum survival time of 12 months. Due to this experience, the development focused on the combination of clear optics with surrounding haptics, and in 1900 Salzer reported on the survival of 2,5 years of a quartz optic with horn haptic. The development of alloplastic keratoplasties stopped when Zirm reported on successful kerastoplasty with homologues implants in 1906.

It was not before 1940 when Wünsche re-initiated experiments with alloplastic material: Polymethylmethacrylate (PMMA). The publications of Ridley and Roper-Hall about the intraocular biocompatibility of PMMA encouraged the use of this material in keratoplasty. Best results were achieved with penetrating implants with an optic made of PMMA and various designs of circular haptic intended to fix the optic on or in the cornea (K. Hille, Keratoprothesen—Historischer Überblick, Materialien and Stand der gegenwärtigen Forschung, Ophthalmologe 99, 513-522, 2002). FIG. 1 (a) shows an epiendocorneal (‘nut-and-bolt’), (b) an endostromal, and (c) an epicorneal keratoprosthesis. The epicorneal prosthesis is most frequently covered with an oral mucous transplant.

The long-term results with these PMMA keratoprostheses were generally disappointing. Complications included the melting of tissues next to the haptic, leakage resulting in infections, aseptic inflammation, and epithelialization of the surface of the optic resulting in opacification and rejection. Moreover, there was a high incidence of vitreous inflammation and glaucoma.

In the early sixties Strampelli developed a keratoprothesis using biological material for the haptic. For his ‘osteoodontokeratoprothesis’ (OOKP) Strampelli removed a single-rooted tooth together with the surrounding alveolar bone from the patient's mouth and used this for preparing the haptic. He cut a precision hole into this haptic and fixed a long cylindrical optic made of PMMA into this hole by glue (FIG. 2). It turned out that the rejection rate of Strampelli's implant is rather low, and it is, therefore, by leading ophthalmosurgeons since 40 years considered as the ultima ratio for patients where a keratoplasty with a donor cornea is impossible (K. Hille, Keratoprothesen—Klinische Aspekte, Ophthalmologe 99, 523-531, 2002). However, this implant has a number of shortcomings. It requires three surgical operations. The patient still needs to have a vital single root tooth available for removal together with the surrounding alveolar bone. The implant needs to undergo a three months conditioning phase by placing it in a subcutaneous bag at the lower lid. The cylindrical optic penetrates deeply into the anterior chamber of the eye. The iris and lens have to be removed in order to prevent the growth of retroprosthetic membranes and the development of secondary glaucoma. Due to the size and rigidity of the implant conventional screening of glaucoma is impossible leaving the risk of loss of vision due to glaucoma. And the field of vision is narrow due to the long narrow cylindrical optic. On the other hand this implant has turned out to be tolerated over many years even by patients with dry eyes and a poor prognosis for corneal transplants.

For patients without a single-rooted tooth Pintucci modified trampelli's keratoprosthesis design in 1979 by replacing the dental root with alveolar bone by a soft, pliable Polyethyleneterephthalat (Dacron) tissue made of fabrics. Just like the Strampelli implant the Dacron felt of Pintucci's keratoprosthesis requires preconditioning to allow the ingrowth of tissue in the Dacron felt. The keratoprosthesis with the preconditioned Dacron haptic is then implanted by covering the haptic on top of the cornea with oral mucous tissue and suturing it to the cornea. Oral mucous tissue is preferably used because of its mechanical strength, strong vascularization and fast cellular turn-over.

Recent efforts to develop improved keratoprostheses focus on the use of flexible, biocompatible, porous haptic materials for example from Polytetrafluorethylene (PTFE), Polyethyleneterephthalat (Dacron) or poly-2-Hydroyethylmethacrylate (pHEMA). However the long-term integration of these haptic materials into the body tissues was so far not satisfactory. Also attempts to replace the rigid optic with flexible silicone or poly-2-Hydroyethylmethacrylate (pHEMA) were not very successful.

Out of the currently available keratoprostheses the Strampelli design has the best long-term clinical success, followed by the Pintucci design. Both keratoprosthesis designs require a long optical cylinder with a small diameter in order to prevent the growth of retroprosthetic membranes. This means that both the iris and lens have to be removed and the patient will have a small visual field.

Object of the invention is to provide a keratoprosthesis which has an improved visual field and can be implanted easily.

The object is solved by a keratoprosthesis in which the chemical nature of the posterior surface of the optic part is rendered such that the formation of a retroprosthetic membrane is prevented allows to significantly reduce the length of the optical cylinder thus enlarging the visual field and allowing the patient's iris and lens to remain in place.

The intraocular pressure exercised on the unsupported optical part of the keratoprosthesis must be born by the haptic part attached to the remaining cornea. The force exercised by the intraocular pressure is proportional to the cross section of the optic, that means proportional to the square of the optic diameter. Therefore, the optic diameter shall be as small as reasonably possible, whereas the overall diameter of the keratoprosthesis should be large in order to distribute the forces across a large haptic area.

A rigid haptic has the disadvantage of not following the movement of corneal tissues thus causing local mechanical stress. A flexible haptic will follow the movement of the surrounding corneal tissue and prevent local stress. In a preferred embodiment the rigidity of the haptic material is similar to the corneal tissue, i.e. mimic the material. If the haptic material is too soft the haptic will exercise strong radial forces Fr not evenly distributed over the corneal tissue and cause shear stress.

Both optic and haptic material is hydrophobic, i.e. absorb less than 5 percent water in order to avoid interaction with eye medications and dimensional changes due to changes in hydration. Preferrably both optic and haptic are made of the same flexible optically clear polymer. Alternatively the flexible haptic polymer can be polymerized as an interpenetrating network to the optic polymer which then may be rigid.

The front portion of the optic not in contact with corneal tissue shall preferably be coated by a hydrophilic layer adsorbing water, enable a smooth gliding of the eye lid and support the spreading of artificial tear when instilled in the eye.

The haptic shall enable the anchoring and ingrowth of surrounding tissue including vascularization. In order to enable this the topograghy of surface can be textured with ridges, groves, pillars, cylindric holes, pores, mesh structure, spikes or similar. In order to maximize cellular attachment and minimize inflammatory response the surfaces of the keratoprosthesis in contact with tissue has preferably a cell adhesive biochemical coating such as fibronecting and/or use the topography-associated surface free energy to promote cell adherence. Pores or holes preferably penetrate the haptic in order to allow vascularization.

By creating keratoprostheses with such biomimetic characteristics the most serious drawbacks of the known designs could be overcome, not requiring a several step surgical procedure with preconditioning of the haptic part.

### Embodiments of the Invention

Coating of PMMA with Heparin

The surface of clinical quality Polymethylmethacrylate (PMMA) platelets was saponified by incubation in 3 M Sodium hydroxide at 70° C. for 24 hours. Thereby a negatively charged surface was created. After washing the activated PMMA platelets with Sodium carbonate buffer solution at pH 9 the surface was coated with Polyethyleneimine (PEI) by ionic bonding. The successful coating with PEI was verified by staining with Eosin red solution. Sodium heparine was activated by Sodium nitrite solution. The PEI coated PMMA platelets were incubated with activated Heparin solution. Subsequently the binding of the activated Heparin to the PEI coated PMMA surface was initiated by incubation with Sodium borohydride solution. The successful Heparin binding was verified by staining with Toluidine blue solution.

In vitro cell adherence tests showed that the heparinized PMMA surfaces strongly inhibited the adherence of human fibroblasts as compared to untreated PMMA surfaces, confirming the potential for preventing retroprosthetic membrane formation on the optical part of keratoprostheses by Heparin coating.

### One-Piece Keratoprotheses

Cylindric polymer buttons of poly-Phenoxyethylacrylate (POEA) with 20 mm diameter and 5 mm height were obtained by thermal polymerization of Phenoxyethylacrylate in closed moulds with N,N-Azobisisobutyronitril (AIBN) as initiator and Ethyleneglycoledimethacrylate (EGDMA) as crosslinker.

Commercially available buttons of a copolymer of Laurylmethacrylate (LMA), Methylmethacrylate (MMA) and 2-Ethoxyethylmethacrylate (EOEMA) were obtained from Benz Research & Development Corporation, Sarasota, Fl., USA (BENZ CLEAR HYDROPHOBIC HF-1 material).

Both polymers are hydrophobic acrylic polymers with a glass transition temperature of approximately 10° C. At the corneal temperature (˜35° C.) both materials are flexible with their rigidity similar to corneal tissue.

One-piece prototype keratoprostheses for epicorneal and endocorneal implantation were manufactured by cryo-milling and cryo-lathing from both materials with larger, more stable holes for suturing and smaller holes for tissue ingrowth (FIG. 5 shows an example).

### Coating of POEA

The surface of POEA was activated (ionized) by treatment with Argon plasma.

In order to permanently render the outer surface of a keratoprosthesis (which is in contact with tearfilm) strongly hydrophilic, a solution of 2,3 Dihydroxypropylmethacrylate (DHPMA) and UV initiator was sprayed on the activated POEA surface and polymerized by UV light. The DHPMA coated POEA surface was stable against light, hydrolysis and aging. It allowed perfect spreading of water.

For creating bioactive surfaces the activated POEA surface was coated by a layer-by-layer technique three times alternatively depositing Chitosan from crab shells (Chi) and Heparin sodium salt (Hep). The Chi-Hep-Chi-Hep-Chi-Hep coating resulted in a stable Heparin coating of the POEA.

In another experiment an additional layer of Fibroblast Growth Factor (FGF) was bound to the outer Heparin layer resulting in FGF coated POEA.

Coating of HF-1

The surface of HF-1 polymer was activated (ionized) by treatment with Nitrogen plasma.

In order to permanently render the outer surface of a keratoprosthesis (which is in contact with tearfilm) strongly hydrophilic, a solution of 2,3

Dihydroxypropylmethacrylate (DHPMA) and UV initiator was sprayed on the activated HF-1 surface and polymerized by UV light. The DHPMA coated HF-1 surface was stable against light, hydrolysis and aging. It allowed perfect spreading of water.

For creating bioactive surfaces the activated HF-1 surface was coated by a layer-by-layer technique two times alternatively depositing Chitosan from crab shells (Chi) and Heparin sodium salt (Hep). The Chi-Hep-Chi-Hep coating resulted in a stable Heparin coating of the HF-1 polymer.

In another experiment an additional layer of Fibronectin-like Engineered Protein Polymer (FEPP), a peptide known to promote cell adherence, was bound to the outer Heparin layer resulting in FEPP coated HF-1 polymer.

### Biological Response to Coated Polymers

Coated and uncoated polymers were tested for adherence and proliferation of adherent corneal cells in vitro.

DHPMA coated POEA and HF-1 as well as uncoated HF-1 did not promote cell adherence or proliferation.

Heparin coated POEA and HF-1 resulted in poor cell adherence and proliferation.

Untreated POEA, FGF coated POEA and FEPP coated HF-1 polymer resulted in excellent cell adherence and proliferation.

A series of prototype keratoprostheses with geometric design similar to FIG. 5 were manufactured from HF-1 material. The outer surface of the optic was coated with DHPMA, the sides of the optic and the haptic were coated with FEPP, and the inner surface of the optic was left untreated. These keratoprostheses were implanted in eight rabbit eyes. The rabbit cornea was trepanated to accept the optic of the keratoprosthesis, the keratoprosthesis was placed with the haptic on the cornea (epicorneal) and sutured to the cornea. Then the keratoprosthesis was covered with the rabbit's nictating membrane. After eight weeks observation time the inner and outer optic were completely clear without any fibrin adherence or retroprosthetic membrane formation, and the eyes did not show inflammatory responses.

### BRIEF DESCRIPTION OF THE DRAWINGS

Other objects, advantages and novel features of the present invention will become apparent from the following detailed description when considered in conjunction with the accompanying drawings herein.

FIGS. 1a-1c are schematic showings of known examples of the keratoprosthesis;

FIG. 2 is a showing of a known keratoprosthesis;

FIGS. 3a and 3b are schematic views of optical cylinders of keratoprostheses;

FIG. 4 shows an embodiment of an inventive keratoprosthesis in side view;

FIG. 5a is a sectional view of the embodiment of FIG. 4 along the section line A-A in FIG. 5b; and

FIG. 5b is a plan view of the embodiment showing FIGS. 4 and 5a.

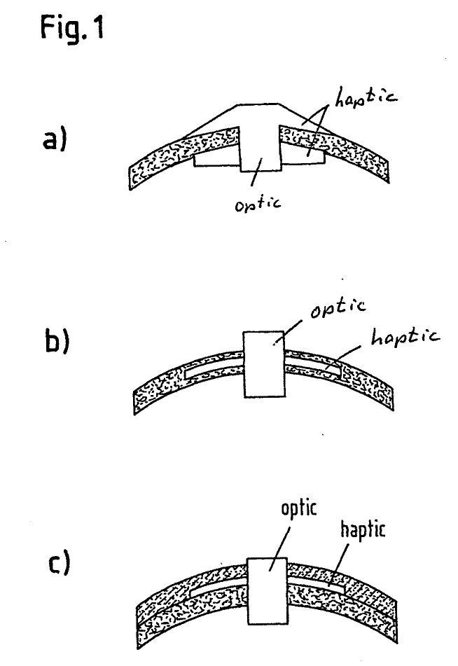

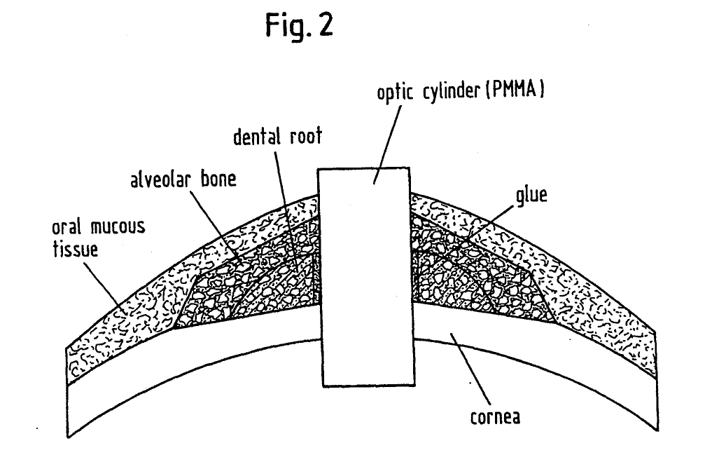

### DETAILED DESCRIPTION OF THE DRAWINGS

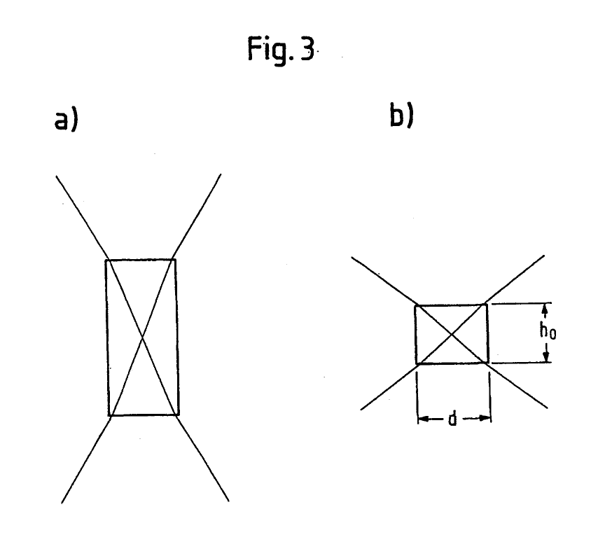

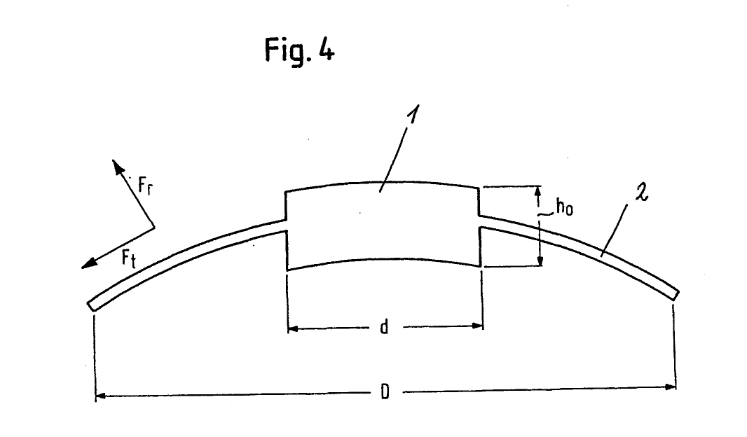

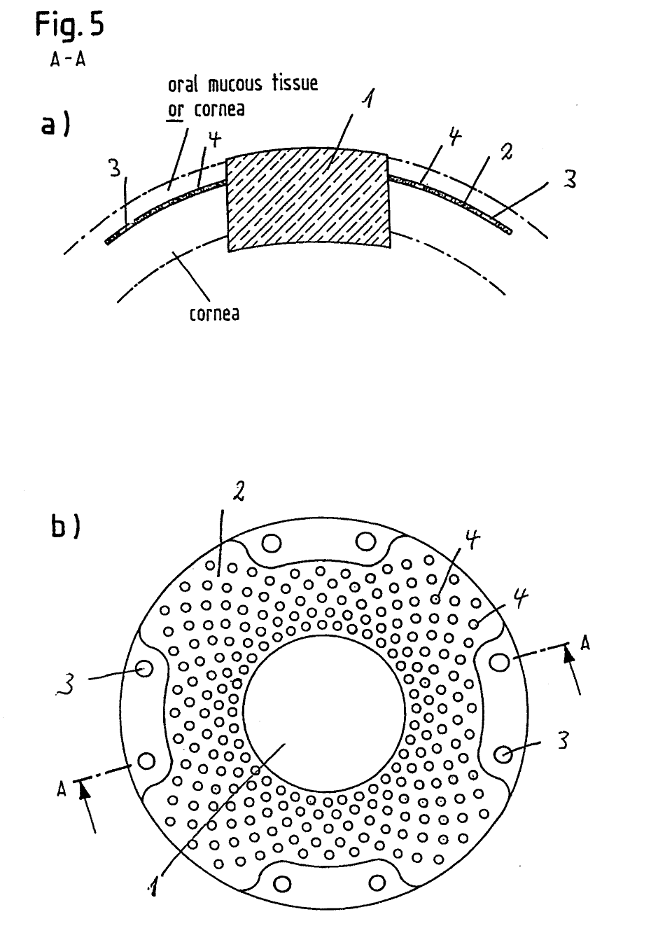

Out of the currently available keratoprostheses the Strampelli design has the best long-term clinical success, followed by the Pintucci design. Both keratoprosthesis designs require, however, a long optical cylinder (length ho) with a small diameter (d) in order to prevent the growth of retroprosthetic membranes (FIG. 3a). This means that both the iris and lens have to be removed and the patient will have a small visual field. Rendering the chemical nature of the posterior surface of the optical cylinder 1 such that the formation of a retroprosthetic membrane is prevented allows to significantly reduce the length ho of the optical cylinder 1 thus enlarging the visual field (FIG. 3b) and allowing the patient's iris and lens to remain in place. FIGS. 4, 5(a) and 5(b) show a one-piece keratoprosthesis for epicorneal and endocorneal implantation larger more stable holes, for suturing and smaller holes, for tissue ingrowth.

The intraocular pressure exercised on the unsupported optical cylinder 1 of the keratoprosthesis is born by the haptic 2 attached to the remaining cornea. The force exercised by the intraocular pressure is proportional to the cross section of the optic, that means proportional to the square (d²) of the optic diameter (d). Therefore, the optic diameter shall be as small as reasonably possible, whereas the overall diameter (D) of the keratoprosthesis should be large in order to distribute the forces across a large haptic area (FIGS. 4, 5(a) and 5(b)).

The flexible haptic 2 will follow the movement of the surrounding corneal tissue and prevent local stress. Ideally the rigidity of the haptic material should be similar to that of the corneal tissue, i.e. mimic the material. If the haptic material is too soft the haptic will exercise strong radial forces Fr not evenly distributed over the corneal tissue and cause shear stress.

Preferrably both the optical cylinder 1 and the haptic 2 are made of the same flexible optically clear polymer. Alternatively the flexible haptic polymer can be polymerized as an interpenetrating network to the optic polymer which then may be rigid.

The front portion of the optical cylinder not in contact with corneal tissue can preferably be coated by a hydrophilic layer adsorbing water, enable a smooth gliding of the eye lid and support the spreading of artificial tear when instilled in the eye.

The haptic preferably enables the anchoring and ingrowth of surrounding tissue including vascularization. In order to enable this the topograghy of haptic surface can be textured with ridges, groves, pillars, cylindric holes, pores, mesh structure, spikes or similar, and the haptic form a scaffold to support tissue ingrowth. In order to maximize cellular attachment and minimize inflammatory response the surfaces of the keratoprosthesis in contact with tissue have a cell adhesive biochemical coating such as fibronecting and/or use the topography-associated surface free energy to promote cell adherence. The pores or holes 4 in FIG. 5b penetrate the haptic in order to allow vascularization. The haptic 2 of the embodiment shown in FIG. 5c includes pores or holes 4 and circularly bent slits 5 which penetrate the haptic material as well. The slits 5 are arranged along circles on the haptic 2.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1. Keratoprosthesis having of a central optical part tightly connected to a peripheral haptic part both made of hydrophobic polymers comprising:
smooth surfaces of the optical part where the surface oriented toward the eyelid has been rendered hydrophilic, whereas, the chemical nature of the surface oriented to the anterior chamber of the eye is such that it does not support fibrin adherence and membrane formation, wherein
the chemical nature of the textured haptic part is configured to promote the adherence of cells and eventually the vascular ingrowth.

2. Keratoprosthesis according to claim 1, wherein the haptic material has a rigidity similar to that of the corneal tissue of the eye.

3. Keratoprosthesis according to claim 1, wherein the haptic material is flexible and polymerized as an interpenetrating network to the optic material which is a rigid material.

4. Keratoprosthesis according to claim 2, wherein the haptic material is flexible and polymerized as an interpenetrating network to the optic material which is a rigid material.

5. Keratoprosthesis according to claim 1, wherein the optical part has a cylindrical form.

6. Keratoprosthesis according to claim 2, wherein the optical part has a cylindrical form.

7. Keratoprosthesis according to claim 3, wherein the optical part has a cylindrical form.

8. Keratoprosthesis according to claim 1, wherein the keratoprosthesis is one-piece.

9. Keratoprosthesis according to claim 1, wherein a retroprosthetic surface of the optical part is coated by Heparin.

10. Keratoprosthesis according to claim 1, wherein the keratoprosthesis comprises material of an acrylic polymer with a glass transition temperature of approximately 10° C. and having a rigidity similar to that of the cornea tissue at the corneal temperature of about 35° C.

11. Keratoprosthesis according to claim 10, wherein the keratoprosthetis material is one of POEA and a copolymer of LMA, MMA and EOEMA.

12. Keratoprosthesis according to claim 1, wherein the haptic material is coated with one of FGF and FEPP.

13. Keratoprosthesis according to claim 1, wherein the haptic material is provided with penetrating bores or holes.

14. Keratoprosthesis according to claim 1, wherein the haptic material is provided with bores or holes as well as circularly bent slits.
